(19) United States
(12) Patent Application Publication
(10) Pub. No.: US 2020/0363400 A1
(43) Pub. Date: Nov. 19, 2020

(54) Competitive Ligand Binding Assays

(71) Applicant: Regeneron Pharmaceuticals, Inc., Tarrytown, NY (US)

(72) Inventors: Michael A. PARTRIDGE, Tarrytown, NY (US); Giane Oliveira SUMNER, Tarrytown, NY (US); Elif Kabuloglu KARAYUSUF, Tarrytown, NY (US)

(21) Appl. No.: 16/872,767

(22) Filed: May 12, 2020

Related U.S. Application Data
(60) Provisional application No. 62/846,872, filed on May 13, 2019.
(60) Provisional application No. 62/859,914, filed on Jun. 11, 2019.

(51) Int. Cl. G01N 33/50; G01N 33/543; G01N 33/533; G01N 33/68

(52) U.S. Cl. CPC G01N 33/5047; G01N 33/54326; G01N 2458/40; G01N 33/6854; G01N 33/533

(57) ABSTRACT

Improved assays for the detection and optionally the quantification of anti-drug antibodies (ADAs) in a sample are provided. The disclosed assays include a protein drug capture assay format and a protein drug target capture assay format, each of which have certain advantages over existing assays.

### CROSS-REFERENCE TO RELATED APPLICATIONS

This application claims benefit of and priority to U.S. Provisional Patent Application No. 62/846,872 filed on May 13, 2019, and U.S. Provisional Patent Application No. 62/859,914 filed on Jun. 11, 2019, both of which are incorporated by reference in their entirety.

### TECHNICAL FIELD OF THE INVENTION

Aspects of the invention are generally directed to assays for detecting drug interactions, in particular to assays for detecting anti-drug antibodies in samples.

### BACKGROUND OF THE INVENTION

Administration of biological therapeutics to a patient can induce an undesirable immunogenic response in the patient that can lead to the development of anti-drug antibodies (ADA) (Mire-Sluis, A. R., et al., J Immunol Methods, 289(1):1-16 (2004)). Neutralizing antibodies (NAbs) are a subset of ADA that inhibit binding of the drug to its target, rendering the drug biologically inactive. By definition NAbs neutralize the effect of the drug, potentially reducing clinical activity. In addition, where the drug is a biological mimic of an endogenous protein, NAbs may cross react with the drug's endogenous analogue which can have critical consequences for drug safety (Finco, D., et al., J Pharm Biomed Anal, 54(2):351-358 (2011); Hu, J., et al., J Immunol Methods, 419:1-8 (2015)).

Detection of an immunogenic response involves a tiered approach where a sample is first tested for the presence of ADA, typically using a bridging immunoassay (Mire-Sluis, A. R., et al., J Immunol Methods, 289(1):1-16 (2004)). Further characterization of the ADA response may include a titer assay to determine the relative amount of ADA, and an assay to determine whether the antibody response is neutralizing (Wu, B., et al., AAPS Journal, 18(6):1335-1350 (2016); Shankar, G, et al., J Pharm Biomed Anal 48(5):1267-1281 (2008); Gupta, S., et al., J Pharm Biomed Anal, 55(5):878-888 (2011)).

NAb assays are usually very sensitive to the presence of drug in the sample (Xu, W., et al., J Immunol Methods, 462:34-41 (2018); Xu, W., et al., J Immunol Methods, 416:94-104 (2015); Xiang, Y., et al., AAPS Journal, 21(1):4 (2019); Sloan, J. H., et al., Bioanalysis, 8(20):2157-2168 (2016)). The tolerance for drug in NAb assays is generally lower than the ADA assays that initially detect the immunogenic response, and often lower than the trough concentrations of drug in patients. Therefore, some neutralizing antibody responses may not be detected in the NAb assay due to interference from drug in the sample.

A number of approaches for improving drug tolerance (DT) in ADA assays have been reported including acid treatment to dissociate drug:ADA complexes allowing improved detection of the drug, or long sample incubations that allow the labeled drugs in the method to displace the free drug:ADA complexes (Sloan, J. H., et al., Bioanalysis, 8(20):2157-2168 (2016); Patton, A., et al., J. Immunol Methods, 304(1-2):189-195 (2005); Butterfield, A. M., Bioanalysis, 2(12), 1961-1969 (2010)).

Several solid phase extraction or precipitation methods have also been reported that improve DT of ADA assays. Broadly these methods can be divided into two groups: methods that extract the ADA from the sample, and methods that deplete drug from the sample (Zoghbi, J., et al., J Immunol Methods, 426:62-69 (2015); Smith, H. W., et al., Regul Taxicol Pharmacol, 49(3):230-237 (2007); Niu, H., J Immunol Methods, 446, 30-36 (2017); Muram, T. M., et al., J Invest Dermatol, 136(7):1513-1515 (2016); Chen, Y. Q., J Immunol Methods, 431:45-51 (2016); Bourdage, J. S., et al., J Immunol Methods, 327(1-2):10-17 (2007)). Similar approaches have also been applied to improve the DT of NAb assays (Xu, W. et al., J Immunol Methods, 462:34-41 (2018); Xu, W., et al., J Immunol Methods, 416:94-104 (2015); Xiang, Y., et al., AAPS J, 21(1):4, (2018); Xiang, Y., et al., AAPS J 21(3):46 (2019)). In one case the affinity capture elution method was used to isolate and detect ADA, and competitive inhibition with free target was used to identify NAbs (Sloan, J. H., et al., Bioanalysis, 8(20):2157-2168 (2016)).

Competitive ligand binding (CLB) assays are highly reproducible and relatively easy to perform. They are at least comparable, and in some cases superior, to cell based assays with respect to sensitivity, assay variability and matrix interference (Finco, D., et al., J Pharm Biomed Anal, 54(2):351-358 (2011).

Therefore, it is an object of the invention to provide assays that have improved drug tolerance relative to existing assays.

It is another object of the invention to provide improved assays that avoid or eliminate the problem of protein drug carryover.

### SUMMARY OF THE INVENTION

Improved assays for the detection and optionally the quantification of anti-drug antibodies (ADAs) including neutralizing antibodies (NAbs) in a sample are provided. It has been discovered that careful selection of assay reagents mitigates, reduces, or eliminates the carryover problem in existing assays. CLB NAb assays were developed for two drug programs. The methods were optimized for sensitivity and DT, and included an acid dissociation step. In some embodiments assays had DT levels that were substantially lower than the trough levels of drug. The disclosed assays include a drug capture assay format and a drug target capture assay format, each of which have certain advantages over existing assays. In some embodiments, the target capture assays are designed so that free drug is washed away before addition of the labeled target thereby obviating the carryover problem that generates a false positive. In other embodiments the assays are designed so that drug:anti-drug complexes are washed away before adding to the target coated plate. Target interference can potentially be eliminated or minimized with a competing target blocking reagent in the sample incubation step. In an exemplary target capture assay format, a mild acid approach is used to minimize free target interference.

One embodiment provides a drug capture method for detecting anti-drug antibodies, for example NAbs, to a drug in a sample. The drug can be a small molecule or a protein drug. Representative protein drugs include, but are not limited to antibodies, fusion proteins, and therapeutic proteins. One method includes the steps of incubating the sample under acidic conditions for a period of time to produce an acidified sample. In certain embodiments, the sample is obtained from a subject, for example a human subject, prior to administration, after administration, or during treatment with the drug. The acidified sample can have a pH of 2.0 to 4.0. In some embodiments, the acid treatment promotes the dissociation of complexes including, but not limited to, NAb:drug complexes and drug:target complexes. A target of the drug is added to the acidified sample, and the pH of the acidified sample is raised to a neutral pH, for example about 7.0 so that the added target can bind to drug in the sample. In one embodiment, the added target is in a pH buffer such as a Tris buffer when added to the acidified sample. In some embodiments, the target is labeled with a selectable label that aids in the physical removal of complexes formed in the sample that contain the labeled target. Representative selectable labels include, but are not limited to, mass tags, magnetic beads, protein tags, and metallic particles and are discussed in more detail below. Complexes formed in the sample that contain the labeled target are physically removed from the sample to produce a depleted sample. The complexes containing the labeled target include target:drug complexes. Removing the target:drug complexes reduces the concentration of drug in the depleted sample. When the target is labeled with magnetic beads, magnetism is used to physically remove the target:drug complexes to produce the depleted sample. The depleted sample is incubated with an anti-target blocking reagent and labeled drug, for example biotinylated drug, to produce an assay sample. Exemplary anti-target blocking agents include, but are not limited to, antibodies or an antigen binding fragment thereof, receptor molecules, and soluble receptors. In some embodiments, the anti-target blocking reagent is an antibody that specifically binds the target and prevents or inhibits the target from binding to the drug. The assay sample is then incubated on an avidin-coated or a streptavidin-coated solid support. In some embodiments, the solid support is washed after incubation with the assay sample to remove unbound reagents. The method further includes adding labeled target of the drug to the solid support. The target is typically labeled with a detectable label such as a fluorophore, a chemiluminescence probe, an electrochemiluminescence probe, a quantum dot, a rare earth transition metal, gold metal particles, silver metal particles, or a combination thereof. In one embodiment the label is ruthenium. The solid support is optionally washed to remove unbound labeled target. Detectable signal from labeled target bound to the biotinylated drug bound to the solid support is detected and optionally quantified. A decreased amount of signal from the solid support relative to a control sample indicates the presence of anti-drug antibodies in the sample. In some embodiments, the anti-drug antibodies include neutralizing antibodies that specifically bind to the protein drug and inhibit or block target from binding the drug.

In some embodiments, the drug is a monoclonal antibody, a bispecific antibody, an Fab fragment, an F(ab′)₂ fragment, a monospecific F(ab′)₂ fragment, a bispecific F(ab′)₂, a trispecific F(ab′)₂, a monovalent antibody, an scFv fragment, a diabody, a bispecific diabody, a trispecific diabody, an scFv-Fc, a minibody, an IgNAR, a v-NAR, an hcIgG, or a vhH.

In some embodiments, the magnetic label is a paramagnetic label or a superparamagnetic label. The magnetic label can be metallic particle, metallic microparticle, metallic nanoparticle, metallic bead, magnetic polymer, uniform polystyrene spherical beads, or a superparamagnetic spherical polymer particle. In some embodiments agarose/sepharose beads and gravity or a centrifuge are used for separation.

In some embodiments of the drug capture assay, the drug tolerance is at least 3- to 20-fold or 10-fold greater in a depleted sample compared to a non-depleted sample. In other drug capture embodiments, the assay positively identifies NAbs in samples taken from the subject at least 29 days after administration of the drug. In another embodiment, the assay positively identifies NAbs in samples taken 85 days after administration of the drug.

It will be appreciated that the methods disclosed herein optionally include incubation or washing steps in which the sample plate, assay plate, or sample is agitated, for example rotated, to assist in the removal of unbound reagents.

Still another embodiment provides a target capture method for detecting anti-drug antibodies to a drug in a sample. The method includes the steps of incubating the sample under acidic conditions for a period of time to produce an acidified sample. The acid treatment induces or promotes drug:NAb complexes and drug:target complexes to dissociate. In some embodiments the acidified sample has a pH of about 2.0-4.0. The acidified sample is then combined with a pH buffered solution containing a labeled anti-drug antibody specific for the protein drug to produce antibody:protein drug complexes. In this step, the pH of the sample is about 4.0-5.5 to minimize free target interference. In some embodiments, the labeled anti-drug antibody is labeled with a selectable label that aids in the physical separation of complexes containing the labeled anti-drug antibody out of the sample. The labeled anti-drug antibody can be a non-blocking anti-idiotypic antibody or antigen binding fragment thereof. The target capture method includes removing the non-blocking antibody:drug complexes from the sample using the selectable label to produce a depleted sample. Typically the selectable label is a magnetic label used to remove non-blocking anti-idiotypic antibody:drug complexes with a magnet or magnetism. In other embodiments, the selectable label can be mass tags, or agarose beads and gravity or centrifugation can be used to separate the non-blocking antibody:drug complexes from the sample. The target capture method includes incubating the depleted sample with labeled drug at about pH 7.0 to produce an assay sample. The labeled drug will bind to NAb present in the sample. In some embodiments, some of the labeled drug remains unbound. The labeled drug is typically labeled with a detectable label. The detectable label in the target capture method can be a fluorophore, a chemiluminescence probe, an electrochemiluminescence probe, a quantum dot, a rare earth transition metal, gold particles, silver particles, or a combination thereof. The target capture method includes incubating the assay sample on a target-coated solid support, wherein the labeled drug specifically binds the target-coated solid support. The target capture method optionally includes washing the solid support after incubation with the assay sample to remove unbound labeled drug. The target capture method also includes the step of measuring a detectable signal from labeled drug bound to the target-coated solid support, wherein a decreased amount of signal relative to a control sample indicates the presence of anti-drug antibodies in the sample.

In some embodiments of the target capture method, the anti-drug antibodies include neutralizing antibodies that specifically bind to the protein drug. The protein drug can be an antibody or antigen binding fragment thereof or a fusion protein. In some embodiments, the antibody is a monoclonal antibody, a bispecific antibody, an Fab fragment, an F(ab′)₂ fragment, a monospecific F(ab′)₂ fragment, a bispecific F(ab′)₂, a trispecific F(ab′)₂, a monovalent antibody, an scFv fragment, a diabody, a bispecific diabody, a trispecific diabody, an scFv-Fc, a minibody, an IgNAR, a v-NAR, an hcIgG, or a vhH.

As with the drug capture method, the selectable label in the target capture method can be a magnetic label. The magnetic label can be a paramagnetic label or a superparamagnetic label. In some embodiments, the magnetic label is a metallic particle, metallic microparticle, metallic nanoparticle, metallic bead, magnetic polymer, uniform polystyrene spherical bead, or a superparamagnetic spherical polymer particle. In some embodiments, the selectable label can be mass tags or agarose/sepharose beads and gravity or centrifugation can be used to separate drug complexes from the sample.

In some embodiments of the target capture method, the drug is detectably labeled with ruthenium.

Some embodiments of the target capture assay have drug tolerance that is least 10 fold greater in a depleted sample compared to a non-depleted sample. In still other embodiments of the target capture method, the method positively identifies NAbs in samples taken from the subject at least 29 days or at least 85 days after administration of the protein drug.

Another embodiment provides a method for identifying a lead protein drug including the steps of administering one or more protein drug candidates to one or more subjects, performing any one of the methods disclosed herein on one or more samples obtained from the one or more subjects, and selecting the protein drug candidate that produce little or no ADAs that reduce the effectiveness of the drug.

### BRIEF DESCRIPTION OF THE DRAWINGS

FIG. 1A is an illustration of an exemplary embodiment of a drug capture competitive ligand assay showing positive and negative assays. FIG. 1B is an illustration of an exemplary embodiment of a target capture competitive ligand assay showing positive and negative assays.

FIG. 2A is a bar graph of assay signal (counts) for a NAb assay for Drug A in the drug capture format where NAb enrichment was attempted with control (no beads) and biotin-drug (Bio-drug) coupled to streptavidin coated-beads (SA-Beads). FIG. 2B is a bar graph of assay signal (counts) for a NAb assay for Drug A in the drug-capture format where drug removal was attempted with control (no beads) and target-coupled beads. FIG. 2C is a bar graph of assay signal (counts) for a NAb assay for Drug B in the target-capture format where drug removal was attempted with control, anti-idiotype antibody coupled beads, and target-coupled beads.

FIG. 3A is a bar graph showing assay signal (counts) for a NAb assay for Drug A in the drug-capture format where drug removal was attempted with (from left to right for each group of three) control (empty bar), target-beads with anti-target mAb (left hatched bar), and target-coupled beads without anti-target mAb (right hatched bar). FIG. 3B is a bar graph showing assay signal (counts) for a NAb assay for Drug B in the target-capture format where drug removal was attempted with (from left to right for each group of three) control (empty bar), non-blocking anti-idiotype antibody coupled beads (left hatched bar), and target-coupled beads (right hatched bar). FIG. 3C is a line graph of % inhibition in a NAb assay for Drug B in the target-capture format with a blocking antibody (top trace; empty circle) and non-blocking mAbs (bottom trace; hatched circle)(ng/mL).

FIG. 4A is a line graph of % inhibition versus drug (μg/mL) in a NAb assay for Drug B in the target-capture format with control (top trace; empty circle) and drug depleted (bottom trace; hatched circle) samples showing drug interference in a NAb negative sample. FIG. 4B is a line graph of % inhibition versus drug (μg/mL) in a NAb assay for Drug B in the target-capture format with control (trace with increasing slope; empty circle) and drug depleted samples (trace with decreasing slope; hatched circle) showing drug tolerance for a NAb positive sample. FIG. 4C is a line graph of % inhibition versus drug (μg/mL) in a NAb assay for Drug A in the drug-capture format with control (bottom trace; empty circle) and drug depleted (top trace; hatched circle) samples showing drug tolerance for a NAb positive sample. FIG. 4D is a line graph of relative light units (RLU) versus Drug A (mg/mL) in an immunoassay to detect drug in samples spiked with the indicated concentration of Drug A for control (top trace; empty circle) and after drug depletion (bottom trace; hatched circle).

FIG. 5A is a scatter plot of percent inhibition versus drug (ng/mL) for ADA positive samples for the control. FIG. 5B is a scatter plot of percent inhibition versus drug (ng/mL) for ADA positive samples after drug depletion.

FIG. 6A is a scatter plot of percent inhibition versus time point (days) for control (circles) and drug depleted (triangles) for all samples in FIG. 5A. FIG. 6B is a scatter plot of percent inhibition versus time point (days) for control (circles) and drug depleted (triangles) that are NAb negative after drug depletion. FIG. 6C is a scatter plot of percent inhibition versus time point (days) for control (circles) and drug depleted (triangles) that are NAb positive after drug depletion.

FIG. 7A-7A-3 is a schematic of an exemplary drug capture assay. FIG. 7B-7-2 is a schematic of an exemplary target capture assay.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx.+/−10%; in other embodiments the values may range in value either above or below the stated value in a range of approx.+/−5%; in other embodiments the values may range in value either above or below the stated value in a range of approx.+1-2%; in other embodiments the values may range in value either above or below the stated value in a range of approx.+/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

“Protein” refers to a molecule comprising two or more amino acid residues joined to each other by a peptide bond. Protein includes polypeptides and peptides and may also include modifications such as glycosylation, lipid attachment, sulfation, gamma-carboxylation of glutamic acid residues, alkylation, hydroxylation and ADP-ribosylation. Proteins can be of scientific or commercial interest, including protein-based drugs, and proteins include, among other things, enzymes, ligands, receptors, antibodies and chimeric or fusion proteins. Proteins are produced by various types of recombinant cells using well-known cell culture methods, and are generally introduced into the cell by genetic engineering techniques (e.g., such as a sequence encoding a chimeric protein, or a codon-optimized sequence, an intronless sequence, etc.) where it may reside as an episome or be integrated into the genome of the cell.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102(H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32(H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv)(see, e.g., see Pluckthun in The Pharmacology of Monoclonal 5 Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

The term “anti-drug antibody” also referred to as “ADA” refers to an antibody that interferes with the activity of a drug.

The term “neutralizing antibody” or “NAb” refers to a subset of anti-drug antibodies that inhibit binding of the drug to its target thereby rendering the drug partially or wholly biologically inactive. Neutralizing anti-drug antibodies (NAbs) have potentially important consequences for both the efficacy and safety of a biological therapeutic.

The terms “individual,” “subject,” and “patient” are used interchangeably herein, and refer to a mammal, including, but not limited to, humans, rodents, such as mice and rats, and other laboratory animals.

### II. Improved Competitive Ligand Binding Assays

Improved assays for the detection and optionally the quantification of anti-drug antibodies (ADAs) in a sample are provided. The disclosed assays include a drug capture assay format and a drug target capture assay format. Identification of ADAs produced in response to the administration of biological therapeutics into a patient is important to satisfy regulatory requirements related to the production and sale of the biological therapeutics and to identify potential dosage problems that can result due to the production of ADAs in a subject.

In some embodiments, the methods for detecting and/or quantifying ADAs in a sample are based on removing free drug from the sample. In one embodiment of the drug capture format, free drug does not generate a false positive as it is washed away before addition of the labeled target. In one embodiment of the drug capture format, target interference is minimized with a competing target blocking reagent in the sample incubation step. In the target capture assay format, a mild acid approach was used to minimize free target interference.

In some embodiments, the drug is an antibody or antigen binding fragment thereof, or a fusion protein.

The disclosed assays overcome the problem of carryover from solid phase extraction/purification steps which can cause interference in the subsequent assay procedure. This is a particular challenge for competitive ligand binding (CLB) NAb assays due to the low concentration of labeled drug used in these methods (Hu, J., et al., J Immunol Methods. 419:1-8 (2015); Wu, B. W., et al., Competitive Ligand-Binding Assays for the Detection of Neutralizing Antibodies. In: Detection and Quantification of Antibodies to Biopharmaceuticals: Practical and Applied Considerations, Michael G. Tovey (Eds). John Wiley & Sons, Inc., Hoboken, N.J., USA. (2011)). The problem of carryover was mitigated in the disclosed assay methods by using drug-specific proteins in a drug depletion procedure. In some embodiments, the drug specific proteins were selected on the basis that they would not interfere in the subsequent NAb assay. One embodiment uses target coupled with beads, with addition of an anti-target blocking reagent in the NAb assay. Another embodiment uses a non-blocking anti-drug antibody-coupled beads. Drug tolerance (DT) was improved by at least >0-fold in both CLB NAb assays after inclusion of the drug depletion step.

In addition to demonstrating improvement in DT with the monoclonal antibody positive control described in the Examples, ADA positive Drug A clinical study samples with therapeutic levels greater than 500 ng/mL were also tested with and without the drug depletion step. When tested with the drug depletion step, these samples showed a marked increase in NAb positivity, indicating that assays with poor DT may under-report the NAb incidence. Thus, the disclosed assay methods also help solve the problem of the under reporting of NAb incidence by conventional assays.

A. Protein Drugs

In some embodiments, the drug is a protein drug. Protein drugs suitable for the disclosed assays include, but are not limited to antibodies and antigen binding fragments thereof (also referred to as antibody protein drugs). In some embodiments, the antibody protein drug can be a monoclonal antibody, a polyclonal antibody, a bispecific antibody, a trispecific antibody or an antigen binding fragments thereof. Representative antibody fragments include but are not limited to an Fab fragment, an F(ab′)₂ fragment, a monospecific F(ab′)₂ fragment, a bispecific F(ab′)₂, a trispecific F(ab′)₂, a monovalent antibody, an scFv fragment, a diabody, a bispecific diabody, a trispecific diabody, an scFv-Fc, a minibody, an IgNAR, a v-NAR, an hcIgG, or a vhH.

In some embodiments, the protein drug product (protein of interest) is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)₂ fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In one embodiment, the antibody is a chimeric IgG2/lgG4 antibody. In one embodiment, the antibody is a chimeric IgG2/IgG1 antibody. In one embodiment, the antibody is a chimeric IgG2/IgG I/IgG4 antibody.

In some embodiments, the antibody is selected from the group consisting of an anti-Programmed Cell Death 1 antibody (e.g., an anti-PD1 antibody as described in U.S. Pat. No. 9,987,500), an anti-Programmed Cell Death Ligand-1 (e.g., an anti-PD-L1 antibody as described in U.S. Pat. No. 9,938,345), an anti-D14 antibody, an anti-Angiopoetin-2 antibody (e.g., an anti-ANG2 antibody as described in U.S. Pat. No. 9,402,898), an anti-Angiopoietin-Like 3 antibody (e.g., an anti-AngPt13 antibody as described in U.S. Pat. No. 9,018,356), an anti-platelet derived growth factor receptor antibody (e.g., an anti-PDGFR antibody as described in U.S. Pat. No. 9,265,827), an anti-Erb3 antibody, an anti-Prolactin Receptor antibody (e.g., anti-PRLR antibody as described in U.S. Pat. No. 9,302,015), an anti-Complement 5 antibody (e.g., an anti-C5 antibody as described in U.S. Pat. No. 9,795,121), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. No. 9,475,875), an anti-Proprotein Convertase Subtilisin Kexin-9 antibody (e.g., an anti-PCSK9 antibody as described in U.S. Pat. No. 8,062,640 or 9,540,449), an anti-Growth And Differentiation Factor-8 antibody (e.g., an anti-GDF8 antibody, also known as anti-myostatin antibody, as described in U.S. Pat. No. 8,871,209 or 9,260,515), an anti-Glucagon Receptor (e.g., anti-GCGR antibody as described in U.S. Pat. No. 9,657,099 an anti-VEGF antibody, an anti-IL1R antibody, an interleukin 4 receptor antibody (e.g., an anti-IL4R antibody as described in U.S. Pat. Appln. Pub. No. US2014/0271681A 1(now abandoned) or U.S. Pat. No. 8,735,095 or 8,945,559), an anti-interleukin 6 receptor antibody (e.g., an anti-IL6R antibody as described in U.S. Pat. No. 7,582,298, 8,043,617 or 9,173,880), an anti-IL1 antibody, an anti-IL2 antibody, an anti-IL3 antibody, an anti-1L4 antibody, an anti-IL5 antibody, an anti-IL6 antibody, an anti-IL7 antibody, an anti-interleukin 33 (e.g., anti-IL33 antibody as described in U.S. Pat. No. 9,453,072 or 9,637,535), an anti-Respiratory syncytial virus antibody (e.g., anti-RSV antibody as described in U.S. Pat. No. 9,447,173), an anti-Cluster of differentiation 3 (e.g., an anti-CD3 antibody, as described in U.S. Pat. No. 9,657,102 and Appn. Pub. No. US20150266966A1, and in U.S. Application No. 62/222,605), an anti-Cluster of differentiation 20 (e.g., an anti-CD20 antibody as described in U.S. Pat. No. 9,657,102 and Appn. Pub. No. US20150266966A1, and in U.S. Pat. No. 7,879,984), an anti-CD19 antibody, an anti-CD28 antibody, an anti-Cluster of Differentiation-48 (e.g., anti-CD48 antibody as described in U.S. Pat. No. 9,228,014), an anti-Fe dl antibody (e.g., as described in U.S. Pat. No. 9,079,948), an anti-Middle East Respiratory Syndrome virus (e.g., an anti-MERS antibody as described in U.S. Pat. No. 9,718,872), an anti-Ebola virus antibody (e.g., as described in U.S. Pat. No. 9,771,414), an anti-Zika virus antibody, an anti-Lymphocyte Activation Gene 3 antibody (e.g., an anti-LAG3 antibody, or an anti-CD223 antibody), an anti-Nerve Growth Factor antibody (e.g., an anti-NGF antibody as described in U.S. Pat. Appln. Pub. No. US2016/0017029 (now abandoned) and U.S. Pat. Nos. 8,309,088 and 9,353,176) and an anti-Activin A antibody. In some embodiments, the bispecific antibody is selected from the group consisting of an anti-CD3×anti-CD20 bispecific antibody (as described in U.S. Pat. No. 9,657,102 and Appln. Pub. No. US20150266966A1), an anti-CD3×anti-Mucin 16 bispecific antibody (e.g., an anti-CD3×anti-Muc16 bispecific antibody), and an anti-CD3×anti-Prostate-specific membrane antigen bispecific antibody (e.g., an anti-CD3×anti-PSMA bispecific antibody).

In some embodiments, the protein of interest is selected from the group consisting of abciximab, adalimumab, adalimumab-atto, ado-trastuzumab, alemtuzumab, alirocumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cemiplimab, cetuximab, denosumab, dinutuximab, dupilumab, durvalumab, eculizumab, elotuzumab, emicizumab-kxwh, emtansinealirocumab, evinacumab, evolocumab, fasinumab, golimumab, guselkumab, ibritumomab tiuxetan, idarucizumab, infliximab, infliximab-abda, infliximab-dyyb, ipilimumab, ixekizumab, mepolizumab, necitumumab, nesvacumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, sarilumab, secukinumab, siltuximab, tocilizumab, tocilizumab, trastuzumab, trevogrumab, ustekinumab, and vedolizumab.

In some embodiments, the protein of interest is a recombinant protein that contains an Fc moiety and another domain, (e.g., an Fc-fusion protein). In some embodiments, an Fc-fusion protein is a receptor Ec-fusion protein, which contains one or more extracellular domain(s) of a receptor coupled to an Fc moiety. In some embodiments, the Fc moiety comprises a hinge region followed by a CH2 and CH3 domain of an IgG. In some embodiments, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1 RAcP ligand binding region fused to the Il-1R1 extracellular region fused to Fc of hIgG1; see U.S. Pat. No. 6,927,004, which is herein incorporated by reference in its entirety), or a VEGF trap (e.g., aflibercept or ziv-aflibercept, which comprises the Ig domain 2 of the VEGF receptor Flt1 fused to the Ig domain 3 of the VEGF receptor Fikl fused to Fc of hIgG1; see U.S. Pat. Nos. 7,087,411 and 7,279,159). In other embodiments, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

B. Drug Capture Assays

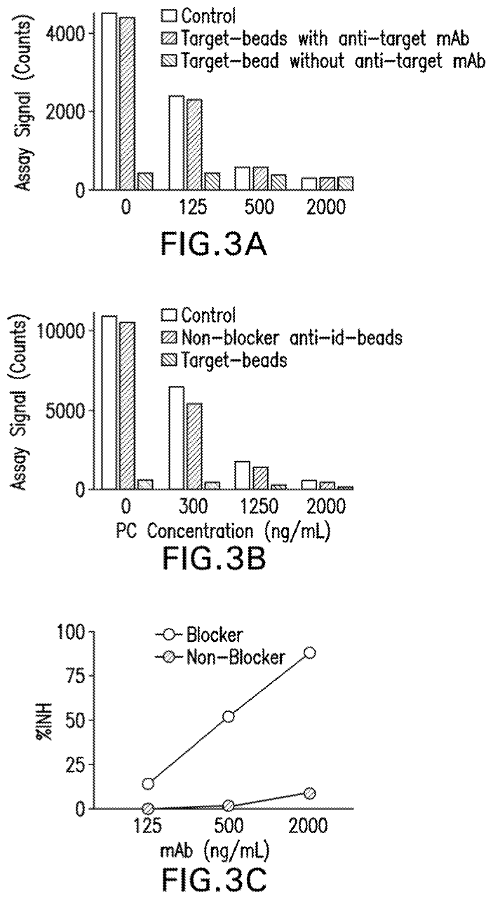

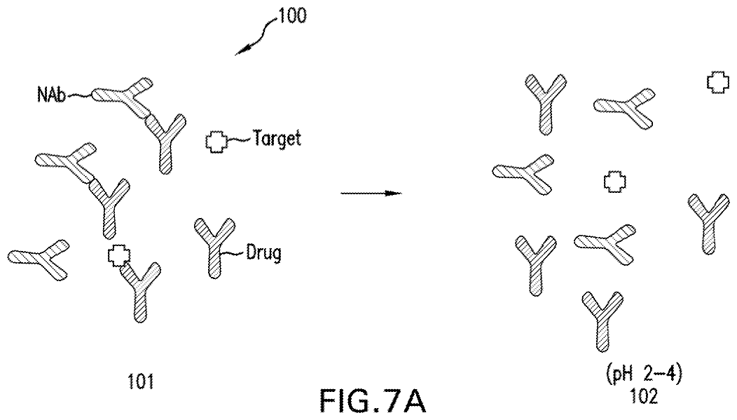

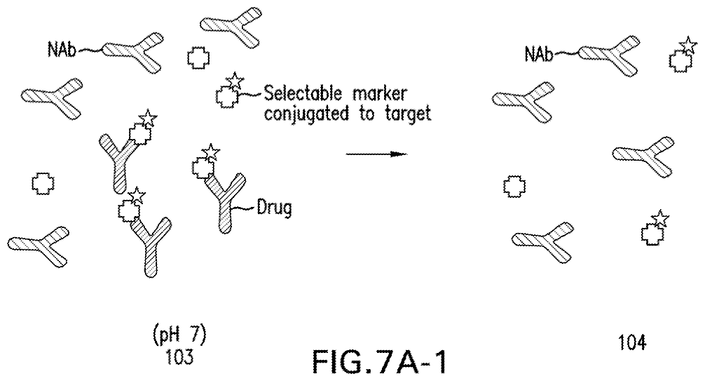

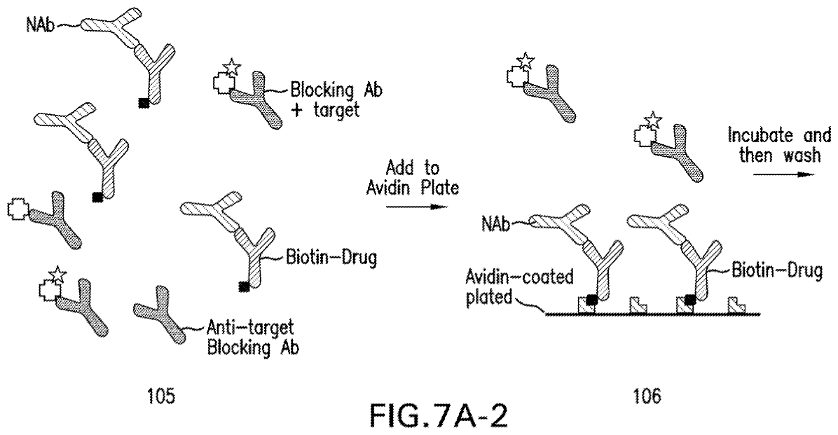

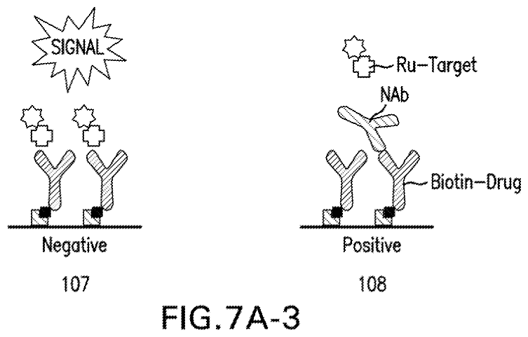

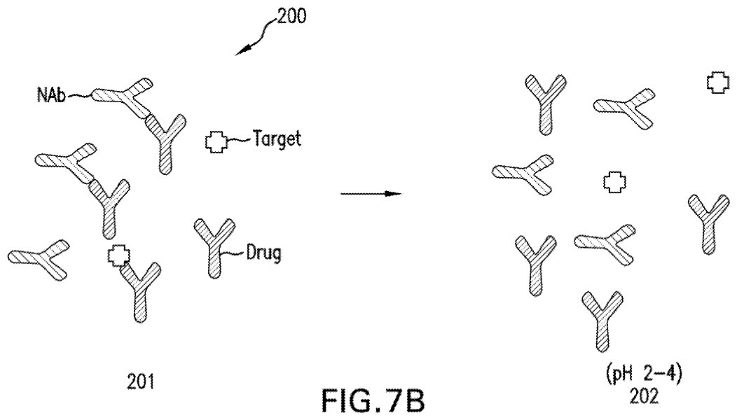

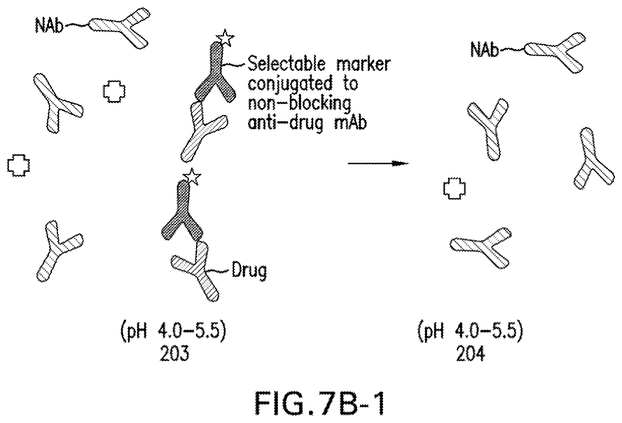

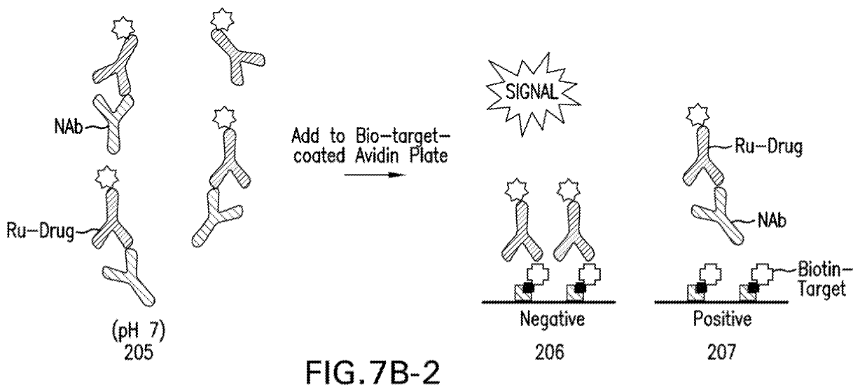

FIG. 7A-7A-3 shows a representative drug capture assay. Assay 100 begins with step 101 in which a sample is obtained from a subject before or after administration of a drug or during treatment with the drug. In this example, the drug is an antibody. Step 101 shows a sample containing drug bound to target, free neutralizing antibodies, and neutralizing antibodies bound to the protein drug antibody. In step 102 the sample is acidified to separate the neutralizing antibodies from the protein drug, and to optionally separate the target from the protein drug.

The sample is acidified to a pH of less than about 5.0, typically to about 2.0 to about 4.0.

In one embodiment the sample is acidified with an acid, for example acetic acid. After acidification, the sample is then incubated at neutral pH, typically about 7.0, with target coupled to a selectable label as shown in step 103. The pH of step 103 can be adjusted to a pH that allows the labeled target to bind to the drug. In some embodiments, the pH is selected so that the NAbs do not bind the drug, but the labeled target can bind the drug.

In one embodiment, the selectable label is a magnetic label, a mass tag, or agarose/sepharose beads. The magnetic label can be a paramagnetic label or a superparamagnetic label. In some embodiments, the magnetic label is a metallic particle, metallic microparticle, metallic nanoparticle, metallic bead, magnetic polymer, uniform polystyrene spherical bead, or a superparamagnetic spherical polymer particle.

The method includes physically removing labeled target:protein drug complexes by exposing the sample to a magnet or magnetic field and isolating the supernatant which is free of labeled target:protein drug complexes to produce a depleted sample. The depleted sample is shown in 104 and contains neutralizing antibodies, optionally free target, and optionally free target coupled to the selectable label. In step 105, biotinylated-drug and an anti-target blocking reagent, for example an antibody that binds free target and target labeled with a selectable marker is added to the sample to form an assay sample. In step 106, the assay sample is then incubated on an avidin-coated or streptavidin coated-solid support, for example an avidin-coated microtiter plate. The plate is optionally washed to remove complexes that do not bind to the avidin-coated plate.

Labeled target is then added to the solid support. The target is typically labeled with a detectable label including a fluorophore, a chemiluminescence probe, an electrochemiluminescence probe, a quantum dot, a rare earth transition metal, gold metal particles, silver metal particles, or a combination thereof. Exemplary fluorophores include but are not limited to Alexa Fluor dyes, Atto labels, CF dyes, Fluorescein Fluorophores, Fluorescent Red, Fluorescent Orange, Rhodamine and derivatives, and Phycobili proteins. In one embodiment, the target is labeled with ruthenium. Signal form the microtiter plate is then detected and optionally quantified. Step 107 shows a strong signal is detected in the absence of NAbs. Step 108 shows a reduced signal in the presence of NAbs.

It will be appreciated that the incubation steps of the method can be followed by one or more wash steps to remove unbound reagents.

One embodiment provides a drug capture method for detecting anti-drug antibodies to a drug in a sample including the steps of incubating the sample under acidic conditions for a period of time to produce an acidified sample, and then combining the acidified sample with a pH buffered solution containing a target of the drug. The target of the drug binds to the drug to produce target:drug complexes. In one embodiment the drug is an antibody or antigen binding fragment thereof or a fusion protein. In some embodiments, the target of the drug is labeled with a selectable label, for example magnetic beads. In this embodiment, the method includes using magnetism to remove the target:drug complexes to produce a depleted sample. The depleted sample is incubated with an anti-target blocking antibody or an antigen binding fragment thereof and labeled drug to produce an assay sample. The anti-target blocking reagent is typically an antibody that specifically binds the target and prevents or inhibits the target from binding to the protein drug. The drug is labeled with a material that allows the labeled drug to be bound to a solid support. An exemplary label is biotin. The assay sample is then incubated on an avidin-coated solid support. In some embodiments, the solid support is washed after incubation with the assay sample to remove unbound reagents. The method further includes adding labeled target of the protein drug to the solid support. The target is typically labeled with a detectable label, for example ruthenium. The solid support is optionally washed to remove unbound labeled target. Detectable signal from labeled target bound to the biotinylated drug bound to the solid support is detected and optionally quantified. A decreased amount of signal from the solid support relative to a control sample indicates the presence of anti-drug antibodies in the sample. In some embodiments, the anti-drug antibodies include neutralizing antibodies that specifically bind to the protein drug.

In some embodiments, the protein drug is a monoclonal antibody, a bispecific antibody, an Fab fragment, an F(ab′)₂ fragment, a monospecific F(ab′)₂ fragment, a bispecific F(ab′)₂, a trispecific F(ab′)₂, a monovalent antibody, an scFv fragment, a diabody, a bispecific diabody, a trispecific diabody, an scFv-Fc, a minibody, an IgNAR, a v-NAR, an hcIgG, or a vhH.

C. Target Capture Assays

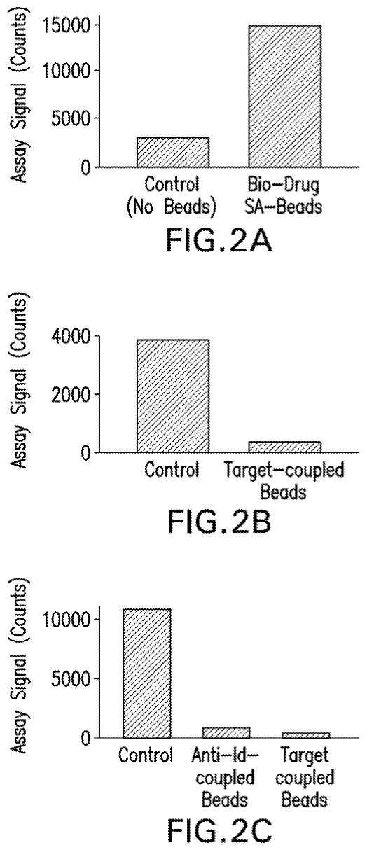

Another embodiment provides a target capture assay. FIG. 7B-7B-2 shows an exemplary target capture assay. Assay 200 begins with step 201 in which a sample is obtained from a subject before or after administration of a drug or during treatment with the drug. In this example, the drug is an antibody. Step 201 shows a sample containing drug bound to target, free neutralizing antibodies, neutralizing antibodies bound to the drug, and optionally free target. In this embodiment the sample is acidified to separate the neutralizing antibodies from the protein drug, and to separate the target from the protein drug, thereby producing an acidified sample as shown in step 202.

The acidified sample is acidified to a pH to promote the dissociation of drug with target and of drug with NAbs. In one embodiment, the pH is reduced to less than about 5.0, typically to about 2.0 to 4.0, even more typically to about 3.0 to 3.5. In some embodiments the sample is acidified with an acid, for example acetic acid. After acidification, the sample is then incubated with a non-blocking anti-drug antibody coupled to a selectable label shown in step 203 with an effective amount of buffer, for example a Tris buffer, to raise the pH to a pH that enables the non-blocking anti-drug antibody coupled with the selectable label to bind the drug in the sample. In one embodiment, the pH is raised to about 4.0 to about 5.5, or to 4.5 to 5.0. The non-blocking anti-drug antibody coupled to a selectable label binds to the drug under these conditions and the target bind very poorly to the drug under these conditions.

In one embodiment, the selectable label is a magnetic label. The magnetic label can be a paramagnetic label or a superparamagnetic label. In some embodiments, the magnetic label is a metallic particle, metallic microparticle, metallic nanoparticle, metallic bead, magnetic polymer, uniform polystyrene spherical bead, or a superparamagnetic spherical polymer particle. The selectable label can be mass tags or agarose/sepharose beads and gravity or centrifugation can be used to separate the complexes containing the selectable label from the sample.

This embodiment of the target capture method includes physically removing labeled drug:anti-drug antibody complexes from the sample using the selectable marker. In one embodiment, the selectable marker is a magnetic label and the drug:anti-drug antibody complexes are physically removed by exposing the sample to a magnet or magnetic field and isolating the supernatant which is free of protein drug: anti-protein drug antibody complexes to produce a depleted sample which contains neutralizing antibodies as shown in step 204.

In step 205, labeled drug is added to the sample under pH neutral conditions to produce an assay sample. In some embodiments, the pH of the depleted sample is raised to about pH 7.0 by the addition of a base or buffer, for example a basic Tris buffer. The buffer and the labeled drug can be added at the same time or in succession. The labeled drug can be labeled with a detectable label including but not limited to a fluorophore, a chemiluminescence probe, an electrochemiluminescence probe, a quantum dot, radioisotope, a rare earth transition metal, gold metal particles, silver metal particles, or a combination thereof. Exemplary fluorophores include but are not limited to Alexa Fluor dyes, Atto labels, CF dyes, Fluorescein Fluorophores, Fluorescent Red, Fluorescent Orange, Rhodamine and derivatives, and Phycobili proteins. In one embodiment, the label is ruthenium.

The assay sample having a pH around 7.0 is incubated on a target-coated solid support. In one embodiment, the solid support is coated with avidin or streptavidin. Biotinylated target is bound to the avidin or streptavidin coated plate. The assay sample is incubated on the solid support to permit binding of sample to the plate. The plate is optionally washed to remove unbound reagents, and the remaining signal is detected and optionally quantified. Step 206 shows labeled drug binding to target bound to the solid support and generating a strong signal. Step 207 shows labeled drug bound by NAb preventing the labeled drug from binding to the solid support resulting in a reduced signal. The reduced signal correlates to the presence of NAbs in the untreated sample.

Still another embodiment provides a target capture method for detecting anti-drug antibodies bound to a drug in a sample that includes the steps of incubating the sample under acidic conditions for a period of time to produce an acidified sample, for example at a pH 2.0-4.0. The acidified sample is then combined with a pH buffered solution containing a labeled anti-drug antibody specific for the protein drug to produce antibody:protein drug complexes and raise the pH to about 4.0 to 5.5, typically to 4.5 to 5.0. In some embodiments, the non-blocking anti-idiotypic mAb is labeled with a selectable label. The labeled anti-drug antibody can be a non-blocking anti-idiotypic antibody or antigen binding fragment thereof. The target capture method includes physically removing the antibody:protein drug complexes from the sample using the selectable label to produce a depleted sample. Typically the selectable label is a magnetic label used to remove drug:anti-drug antibody complexes with a magnet. The target capture method includes incubating the depleted sample with labeled drug at a pH of about 7.0 to produce an assay sample. The labeled drug is typically labeled with a detectable label. The detectable label in the target capture method can be a fluorophore, a chemiluminescence probe, an electrochemiluminescence probe, a quantum dot, a rare earth transition metal, radioisotope, gold particles, silver particles, or a combination thereof. The target capture method includes incubating the assay sample on a target-coated solid support, wherein the labeled drug specifically binds the target-coated solid support. The target capture method optionally includes washing the solid support after incubation with the assay sample to remove unbound labeled reagents. The target capture method also includes the step of measuring a detectable signal from labeled drug bound to the target-coated solid support, wherein a decreased amount of signal relative to a control sample indicates the presence of anti-drug antibodies in the sample.

In some embodiments of the target capture method, the anti-drug antibodies include neutralizing antibodies that specifically bind to the protein drug. The drug can be an antibody or antigen binding fragment thereof or a fusion protein. In some embodiments, the antibody is a monoclonal antibody, a bispecific antibody, an Fab fragment, an F(ab′)₂ fragment, a monospecific F(ab′)₂ fragment, a bispecific F(ab′)₂, a trispecific F(ab′)₂, a monovalent antibody, an scFv fragment, a diabody, a bispecific diabody, a trispecific diabody, an scFv-Fc, a minibody, an IgNAR, a v-NAR, an hcIgG, or a vhH.

In some embodiments of the target capture method, the protein drug is labeled with ruthenium.

Some embodiments of the target capture assay have drug tolerance that is least 10 fold greater in a depleted sample compared to a non-depleted sample. In still other embodiments of the target capture method, the method positively identifies NAbs in samples taken from the subject at least 29 days after administration of the protein drug.

### EXAMPLES

### Example 1: Competitive Ligand Binding NAb Assays: Formats and Drug Tolerance

### Materials and Methods

### Materials and Reagents

All solutions, unless otherwise specified, were prepared in assay buffer (1% BSA in 1×PBS). Read Buffer T (4×) was from Meso Scale Discovery (MSD, Gaithersburg, Md.). Glacial acetic acid (17.4 M) was from Thermo Fisher Scientific (Waltham, Mass.). Human and monkey serum was from BiolVT (Westbury, N.Y.). The fully human monoclonal antibody drugs, the fully human competing anti-target A antibody, the recombinant human targets, the monoclonal, neutralizing anti-drug antibodies, used as the positive controls, and the anti-human monoclonal antibodies, were produced by Regeneron (Tarrytown, N.Y.). Labeling of antibodies and targets with biotin using EZ-link Sulfo-NHS-LC-Biotin (Thermo Fisher Scientific), and with ruthenium NHS ester (MSD), was performed according to the manufacturer's instructions. DyNAbeads™ Antibody Coupling Kit was from Thermo Fisher Scientific. Drug-specific protein reagents were coupled to the magnetic DyNAbeads® according to the manufacturer's instructions (30 μg protein/1 mg beads). Multi-array® High Bind Avidin 96 Well plates were from MSD. Trizma base (1.5 M) was from Sigma (St Louis, Mo.). Wash solution was from KPL Inc.

### Equipment

Microplate washer (ELx405) was from BioTek Instruments (Winooski, Vt.) and microplate shaker from VWR (Radnor, Pa.). The QuickPlex SQ 120 reader was from MSD and the SoftMax® Pro application was from Molecular Devices (Sunnyvale, Calif.).

### Magnetic Bead Drug Depletion Procedure

Samples were diluted 1:5 in 300 mM acetic acid and incubated for 60 mins at room temperature (RT). 30 mg of drug-specific protein-coupled DyNAbeads® were resuspended in a 500 mM Tris solution (Sufficient for one plate of samples/QCs, ˜0.6 mg beads/sample). Acidified samples were then diluted 1:2 (1:20 total final dilution) in 1% BSA, 500 mM Tris solution containing protein-coupled DyNAbeads® (1 hr, 700 rpm). Samples were placed against a magnet and beads were allowed to collect on the tube/well walls, and the supernatant transferred to a separate tube/plate.

### Competitive Ligand Binding NAb Assay Procedure

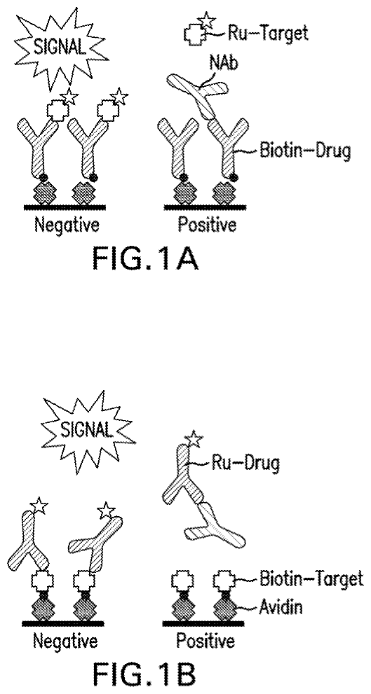

Pooled human serum was used as the Negative Control (NC). Microtiter plates were washed and blocked with 5% BSA for 1 hr at RT. The assay for REGN-A drug was configured in the drug capture format, the assay for REGN-B drug was configured in the target capture format (FIG. 1)(Wu, B. W., ei al., G G, Shankar G: Competitive Ligand-Binding Assays for the Detection of Neutralizing Antibodies. In: Detection and Quantification of Antibodies to Biopharmaceuticals: Practical and Applied Considerations, M. G. Tovey (Ed). John Wiley & Sons, Inc., Hoboken, N.J., USA. (2011)). In both configurations, the labeled drug (biotin or ruthenium) is incubated with the serum sample (with or without drug depletion) in solution prior to adding to the avidin-coated assay plate. In the drug capture format, ruthenium-labeled target is added in a subsequent step, while in the target capture format, biotinylated target is first pre-bound to a streptavidin coated microplate.

### Drug Capture Format:

Samples and QCs (with or without drug depletion) were incubated with 10 ng/mL Bio-REGN-A assay buffer solution containing 50 μg/mL anti-target blocking antibody in a sample plate for 90 mins at RT with shaking (400 rpm). Assay plates were then washed and acidified/neutralized samples and QCs were added (50 μL, 2 hrs RT). Ruthenium labeled recombinant target was added to the assay plate at 2 μg/mL in assay buffer for 1 hr at RT with shaking (50 μL, 400 rpm).

### Target Capture Format:

Biotinylated recombinant target was added to the assay plate at 2 μg/mL in assay buffer for 1 hr at RT with shaking (50 μL, 400 rpm) and washed. Samples and QCs (with or without drug depletion) were incubated with ruthenium labeled drug at 20 ng/mL in assay buffer for 2 hr at RT with shaking (50 μL, 400 rpm). The solution was then added to the assay plate (50 μL, 400 rpm).

In both formats, after the final incubation, plates were washed and 150 μL 2× Read Buffer incubated for 0-10 min and read on a QuickPlex SQ 120 reader. Counts values were imported into SoftMax® Pro software and a plate specific cut point was calculated based on the negative control signal.

### Drug Tolerance Calculations and Cut Point Determination

Drug tolerance (DT) and drug interference values were calculated in SoftMax Pro using a 4PL regression model. Cut points were determined by statistical analysis of data from drug naïve serum samples from diseased individuals tested in the NAb assay. Statistical methods used for the analyses were based on industry practices (Shankar, G., et al., J Pharm Biomed Anal, 48(5):1267-1281 (2008); Gupta, S., et al., J Immunol Methods, 321(1-2):1-18 (2007)).

### Drug A Concentration ELISA

Monkey serum samples were spiked with Drug A at the indicated concentration and then subjected to the drug depletion procedure, or, as a control, to the same processing steps but without addition of target-coupled beads. The resulting serum sample supernatants were then acidified (300 mM acetic acid) and neutralized before adding to a microplate coated with an anti-human Ig, kappa light chain specific mAb. Drug A levels were detected with a biotinylated-anti-human Fc specific mAb, and assay signal generated by NeutrAvidin-HRP.

### Results

CLB NAb assays for two different mAb drugs were developed and optimized for a range of different parameters and including format, sensitivity and DT. For Drug A, a drug capture assay was developed, while for Drug B a target capture method was developed (FIG. 1A-1B). The drug capture CLB NAb assay format is generally preferred because free drug will not generate a false positive response in the absence of NAb, and target interference can be minimized with addition of an anti-target binding protein. However, ruthenium labeled recombinant target for Drug B adhered to the plate even in the absence of biotin-drug, hence a target capture format was selected. In both formats, in the absence of NAbs the labeled drug binds to the labeled target generating a signal in the assay. In the presence of NAbs, binding of the labeled target to labeled drug is inhibited, leading to a reduction in assay signal. Therefore, assay signal is inversely proportional to the amount of NAb in the sample (Wu, B. W., e al., Competitive Ligand-Binding Assays for the Detection of Neutralizing Antibodies. In: Detection and Quantification of Antibodies to Biopharmaceuticals: Practical and Applied Considerations, Michael G. Tovey (Eds). John Wiley & Sons, Inc., Hoboken, N.J., USA. (2011)).

In both NAb assay formats, the presence of free drug in the sample can compete with the labeled drug for binding to NAbs and, in the target capture format, also generating a false positive response in the absence of NAbs. For these reasons DT was a critical variable that required optimization. The assays for both drug programs incorporated an acid dissociation step to improve DT. However, the DT for each method was still substantially lower than the trough drug concentrations in patients (not shown). In one embodiment, the DT for each method was up to 20-fold lower than the trough drug concentrations. Consequently, solid phase extraction/purification methods were assessed to further improve assay DT.

The Drug A clinical study samples were selected based on ADA positivity and drug concentration only, with no consideration for the sampling time point. In samples grouped by sampling time point, NAb positivity was temporal in nature, with 72% of the NAb positive samples occurring at 85 days or later after initial administration. In contrast, 85% of the NAb negative samples were observed at the time points less than 30 days after initial administration. Without addition of the drug depletion step this observation would not be possible in samples containing drug. This is consistent with findings that ADA responses to biologicals and replacement factors mature during the course of treatment, with NAb responses having a higher proportion of IgG4 than is found in normal serum, and the IgG4 response occurring at later time points (Van Schouwenburg, P. A., et al., J Clinical Immunol, 32(5):1000-1006 (2012); Montalvao, S. A., et al., Official J World Federation Hemophilia, 21(5):686-692 (2015); Hofbauer, C. J., et al., Blood, 125(7):1180-1188 (2015); Barger, T. E., et al., European Renal Assoc, 27(2):688-693 (2012)).

### Example II: Carryover of Bead-Coupled Protein to the NAb Assay Step

Materials and Methods

See Example 1.

Results

Initial experiments used a biotin-drug bound to a streptavidin bead to extract the ADA (and NAb) from the sample. However, in CLB NAb assays, low labeled drug concentrations are necessary to achieve maximum sensitivity (Hu, J. et al., J Immunol Methods, 419:1-8 (2015); Wu, B. W., et al., Competitive Ligand-Binding Assays for the Detection of Neutralizing Antibodies. In: Detection and Quantification of Antibodies to Biopharmaceuticals: Practical and Applied Considerations, Michael G. Tovey (Eds). John Wiley & Sons, Inc., Hoboken, N.J., USA. (2011)). Therefore, any biotin-drug transferred from the enrichment step to the assay step could interfere. Data in FIG. 2A demonstrates that biotinylated drug used in the NAb enrichment step carried over to the NAb assay step, resulting in ˜5-fold increase in assay signal. In fact, even in the absence of added biotin-drug in the NAb assay, carryover of biotin-drug from the enrichment step was sufficient to generate substantial assay signal (not shown).

As an alternative to NAb removal from the sample, a drug removal approach was also tested. However, as was the case with biotin-drug NAb enrichment approach, carryover from the protein used to capture the drug could also potentially interfere in the NAb assay step. Data in FIGS. 2B and 2C demonstrates that when using either the target or a blocking anti-idiotype antibody to capture the drug, these proteins carried over and suppressed the signal in the subsequent NAb assay.

### Example III: Minimizing Interference from the Drug Capture Protein

### Materials and Methods

See Example I.

### Results

Attempts to reduce the amount of coupled protein or increasing the wash steps were unable to sufficiently minimize the interference from these proteins ater they were transferred to the NAb assay. In order to mitigate interference from carryover, a different approach was developed for each of the NAb assays.

When target was used as the capture reagent in the drug removal step, the protein carried over and inhibited NAb assay signal (FIGS. 2B, 2C and 3A). However, in the drug capture assay format, addition of an anti-target antibody solved the problem that arises from protein carry over. In the presence of the anti-target mAb, positive and negative control samples subjected the drug removal step with target coupled beads generated assay signal almost identical to control samples without the drug removal step (FIG. 3A).

In the target capture NAb assay format, the addition of anti-target reagent was not possible as it would bind to the capture reagent and inhibited the assay signal. Although a target-blocking anti-idiotype antibody was shown to interfere in the assay (FIG. 2C), it was possible that a non-blocking anti-drug mAb could be used. Therefore, a non-blocking anti-idiotype antibody (FIG. 3C) was coupled to the beads to capture the drug. In this assay format, positive and negative control samples subjected to the drug removal step had similar assay signal to control samples without the drug removal step (FIG. 3B).

### Example IV: Drug Depletion with Magnetic Beads

### Materials and Methods

See Example I.

### Results

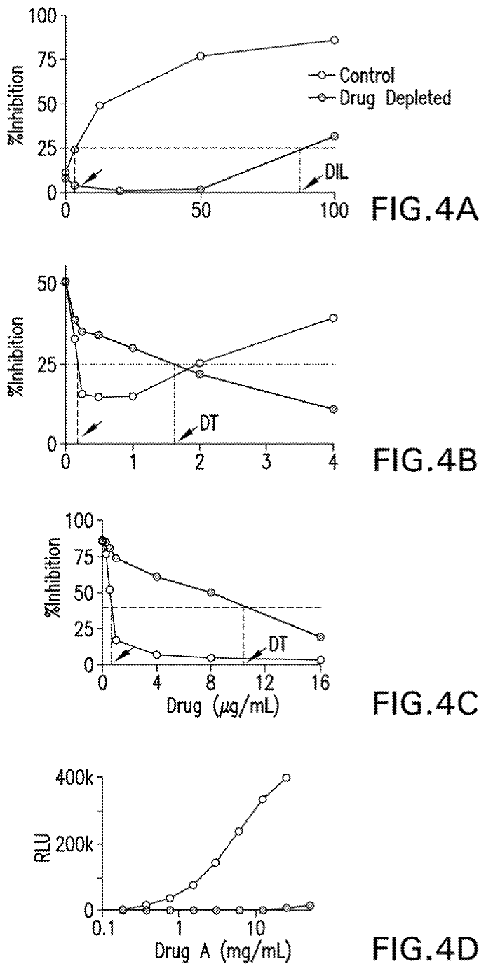

Reducing interference due to protein carried over from the bead step was the key criteria for selecting the specific drug removal reagent. To test the efficiency of drug removal with the protein coupled to the beads, two sets of experiments were performed; drug interference in a NAb negative sample, and DT in a NAb positive sample. To test for drug interference in the target capture assay for Drug B, drug was spiked into a NAb negative sample and tested in the assay with and without the drug depletion step. The addition of the drug removal step with anti-idiotype mAb coupled beads increased the concentration of drug needed to generate a false positive response by almost 50-fold compared to the control (2 μg/mL to 93 μg/mL, FIG. 4A).

To test DT, samples containing the mouse monoclonal positive control antibodies (250 ng/mL) were spiked with increasing concentrations of drug and tested in both assays with and without the drug depletion step. In the target capture assay format for Drug B, the addition of the drug removal step using anti-idiotype mAb coupled beads resulted in a 10-fold increase in DT over the control (153 ng/mL to 1.55 μg/mL, FIG. 4B). With the drug capture format for Drug A, with addition of the drug removal step using target-coupled beads there was a 20-fold increase in DT compared to the control (0.5 μg/mL to 9.7 μg/mL, FIG. 4C). These experiments demonstrated that in both assays DT was substantially improved by incorporation of the drug depletion step.

To determine approximately how much drug was removed by the drug depletion step, monkey serum samples were spiked with Drug A and subjected to the drug depletion procedure. The samples were then analyzed in a sandwich immunoassay with anti-human mAbs as capture and detection reagents. As a control, duplicate Drug A-spiked samples were subjected to the same acidification and neutralization processing steps, but without addition of target-coupled beads. As shown in FIG. 4D. Drug A depleted samples had very poor assay signal compared to the control samples. To quantitate the amount of therapeutic removed, Drug A concentrations in the depleted samples were interpolated from the regression curve generated from the control samples. This analysis demonstrated that approximately 99% of the drug had been removed by the depletion step (e.g., 12.5 μg/mL drug spiked in the sample, 120 ng/mL measured after depletion). Similar levels of depletion were also obtained with the Drug B depletion procedure (not shown).

### Example V: NAb Analysis of ADA Positive Clinical Samples with or without Drug Depletion

### Materials and Methods

The data demonstrates that the drug depletion pre-treatment step improved the DT based on the mouse monoclonal anti-drug antibodies used as the positive controls. To confirm this finding with human anti-drug antibodies, 25 samples from clinical trials with Drug A (multiple-dose) were selected to test in the drug capture NAb assay with and without the drug depletion step

### Results

All samples were ADA positive, and all had detectable drug levels from 500 to 15000 ng/mL. Therapeutic concentrations in all these samples were greater than the DT of the method without the drug depletion step. The ADA response to Drug A was overall quite low (not shown), and the only ADA positive samples that had detectable drug had a low titer response (either minimum dilution or one dilution higher).

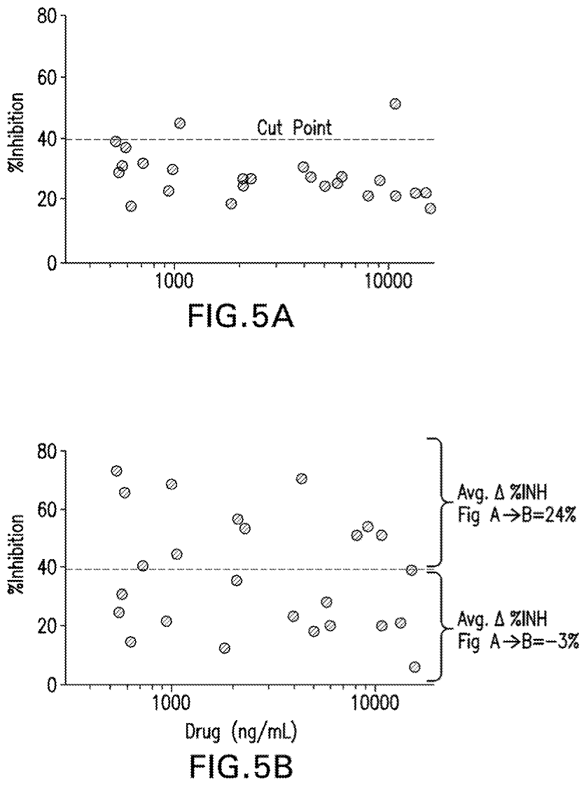

Of the 25 samples tested without the drug-depletion step, only two were NAb positive with % Inhibition greater than the cut point (FIG. 5A). In contrast, after the bead pre-extraction step, twelve samples were NAb positive (FIG. 5B). A selection of ADA negative baseline samples had only very minor changes in % inhibition with or without the bead drug-removal step (not shown). The average change in % Inhibition when samples were analyzed with or without the drug depletion step was +24% for NAb positive samples (FIG. 5B). In contrast, there was no substantial change in % Inhibition for samples that were NAb negative when tested with or without the drug depletion step (−2.7%, FIG. 5B). This clearly indicated that removal of the drug impacted the NAb result for only a subset of samples. Importantly, with the inclusion of the drug depletion step, NAbs were detected when drug concentrations in the samples were up to 10 μg/mL, higher than trough drug levels in the study.

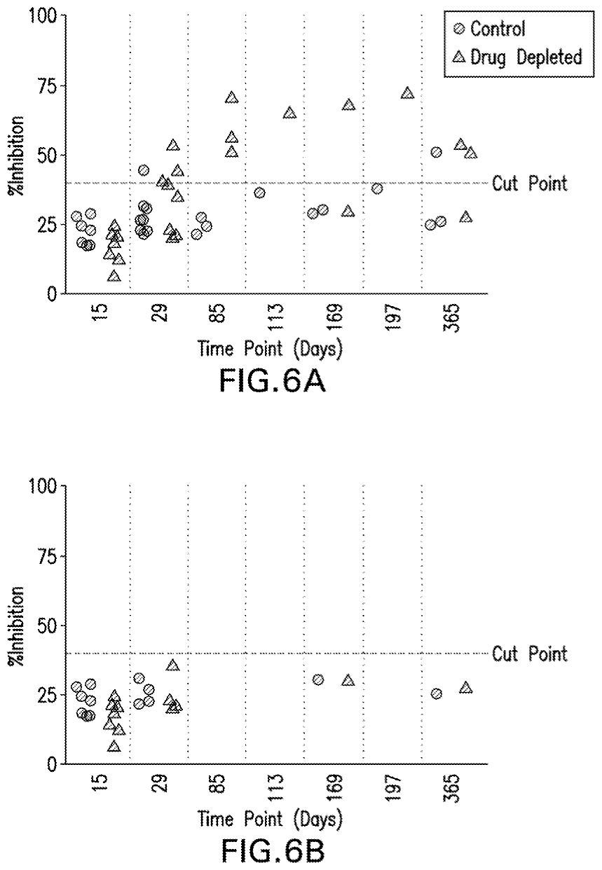

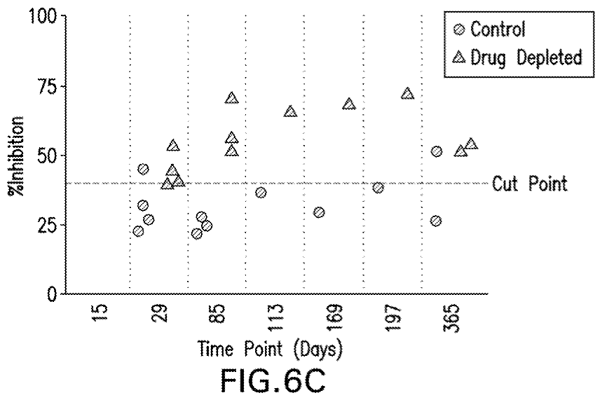

Of the 25 ADA positive samples, the NAb positive samples occur predominantly at later time points (FIGS. 6A-6C). Of the 13 NAb negative samples, 11 (85%) were identified in the two earliest time points tested, Day 15 and 29 after initial administration. In contrast, none of the NAb positive samples were identified at day 15, the earliest time point tested, and only 3 of the 11 NAb positive samples were identified at the next time point tested, day 29. Eight of the 11 NAb positive responses (72%) occurred at time points later than day 85.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

What is claimed is:

1. A method for detecting anti-drug antibodies to a drug in a sample, comprising:
incubating the sample under acidic conditions for a period of time to produce an acidified sample;
combining the acidified sample with a pH buffered solution comprising a target of the drug to produce target:drug complexes, wherein the target of the drug is labeled with a selectable label;
removing the target:drug complexes from the sample using the selectable label to produce a depleted sample;
incubating the depleted sample with an anti-target blocking reagent or an antigen binding fragment thereof and biotinylated drug to produce an assay sample;
incubating the assay sample on an avidin-coated solid support;
optionally washing the solid support after incubation with the assay sample;
adding labeled target of the drug to the solid support;
optionally washing the solid support to remove unbound labeled target; and
measuring a detectable signal from labeled target bound to the biotinylated drug bound to the solid support, wherein a decreased amount of signal from the solid support relative to a control sample indicates the presence of anti-drug antibodies in the sample.

2. The method of claim 1, wherein the anti-drug antibodies comprise neutralizing antibodies that specifically bind to the drug.

3. The method of claim 1, wherein the drug is an antibody or antigen binding fragment thereof or a fusion protein.

4. The method of claim 3, wherein the antibody is a monoclonal antibody, a bispecific antibody, an Fab fragment, an F(ab′)₂ fragment, a monospecific F(ab′)₂ fragment, a bispecific F(ab′)₂, a trispecific F(ab′)₂, a monovalent antibody, an scFv fragment, a diabody, a bispecific diabody, a trispecific diabody, an scFv-Fc, a minibody, an IgNAR, a v-NAR, an hcIgG, or a vhH.

5. The method of claim 1, wherein the acidic conditions comprise a pH of about 2.0 to about 4.0.

6. The method of claim 1, wherein the acidic conditions comprise a pH of 1 to 3.

7. The method of claim 1, wherein the selectable label comprises a magnetic label.

8. The method of claim 7, wherein the magnetic label is a paramagnetic label or a superparamagnetic label.

9. The method of claim 7, wherein the magnetic label is a metallic particle, metallic microparticle, metallic nanoparticle, metallic bead, magnetic polymer, uniform polystyrene spherical beads, or a superparamagnetic spherical polymer particle.

10. The method of claim 1, wherein the anti-target blocking reagent antigen binding fragment thereof specifically binds to the target of the protein drug.

11. The method of claim 1, wherein the drug tolerance of the method is at least 10 fold greater in a depleted sample compared to a non-depleted sample.

12. The method of claim 1, wherein the method positively identifies NAbs in samples containing drug taken from the subject at least 29 days after administration of the protein drug.

13. The method of claim 1, wherein the sample is agitated during the incubation or washing steps.

14. The method of claim 1, wherein the labeled target is labeled with a fluorophore, a chemiluminescence probe, an electrochemiluminescence probe, a quantum dot, a rare earth transition metal, gold metal particles, silver metal particles, or a combination thereof.

15. The method of claim 14, wherein the label comprises ruthenium.

16. A method for detecting anti-drug antibodies to a drug in a sample, comprising:
incubating the sample under acidic conditions for a period of time to promote the dissociation of protein complexes thereby producing an acidified sample;
combining the acidified sample with a pH buffered solution comprising a labeled non-blocking anti-idiotypic antibody specific for the drug to produce non-blocking anti-idiotypic antibody:drug complexes, wherein the labeled non-blocking anti-idiotypic antibody is labeled with a selectable label and the pH is raised to 4.0-5.5;
removing the non-blocking anti-idiotypic antibody:drug complexes from the sample using the selectable label to produce a depleted sample;
incubating the depleted sample with labeled protein drug at a pH of about 7.0 to produce an assay sample;
incubating the assay sample on a target-coated solid support, wherein the labeled drug specifically binds the target;
optionally washing the solid support after incubation with the assay sample to remove unbound labeled drug; and
measuring a detectable signal from labeled drug bound to the target-coated solid support, wherein a decreased amount of signal relative to a control sample indicates the presence of anti-drug antibodies in the sample.

17. The method of claim 16, wherein the anti-drug antibodies comprise neutralizing antibodies that specifically bind to the drug.

18. The method of claim 16, wherein the drug is an antibody or antigen binding fragment thereof or a fusion protein.

19. The method of claim 18, wherein the antibody is a monoclonal antibody, a bispecific antibody, an Fab fragment, an F(ab′)₂ fragment, a monospecific F(ab′)₂ fragment, a bispecific F(ab′)₂, a trispecific F(ab′)₂, a monovalent antibody, an scFv fragment, a diabody, a bispecific diabody, a trispecific diabody, an scFv-Fc, a minibody, an IgNAR, a v-NAR, an hcIgG, or a vhH.

20. The method of claim 16, wherein the acidic conditions comprise a pH of 4.5-5.0 to minimize free target interference.

21. The method of claim 16, wherein the acidic conditions comprise a pH of 2.0-4.0.

22. The method of claim 16, wherein the selectable label comprises a magnetic label.

23. The method of claim 22, wherein the magnetic label is a paramagnetic label or a superparamagnetic label.

24. The method of claim 22, wherein the magnetic label is a metallic particle, metallic microparticle, metallic nanoparticle, metallic bead, magnetic polymer, uniform polystyrene spherical beads, or a superparamagnetic spherical polymer particle.

25. The method of claim 16, wherein the labeled drug is labeled with a fluorophore, a chemiluminescence probe, an electrochemiluminescence probe, radioisotope, a quantum dot, a rare earth transition metal, gold particles, silver particles, or a combination thereof.

26. The method of claim 16, wherein the label comprises ruthenium.

27. The method of claim 16, wherein drug tolerance of the method is at least 10 fold greater in a depleted sample compared to a non-depleted sample.

28. The method of claim 16, wherein the method positively identifies NAbs in samples taken from the subject at least 29 days after administration of the protein drug.

29. The method of claim 16, wherein the sample is agitated during the incubation or washing steps.

30. A method for identifying a lead protein drug comprising:
administering one or more drug candidates to a subject;
performing the method of any one of claim 1 or claim 16 on a sample obtained from the subject; and
selecting the protein drug candidate that produces little or no ADAs.
